(19) United States
(12) Patent Application Publication
(10) Pub. No.: US 2026/0000395 A1
(43) Pub. Date: Jan. 1, 2026

(54) Pressure Distribution Implants for Approximating Opposing Bone Edges of Sternums During Sternal Closure Procedures

(71) Applicant: Ethicon, Inc., Raritan, NJ (US)

(72) Inventor: Barry Inamoto, Lake Barrington, IL (US)

(21) Appl. No.: 18/758,030

(22) Filed: Jun. 28, 2024

(51) Int. Cl. A61B 17/064; A61B 17/00; A61B 17/06

(52) U.S. Cl. CPC A61B 17/0644; A61B 2017/00601; A61B 2017/00619; A61B 2017/06176

(57) ABSTRACT

A pressure distribution implant for use in sternal closure includes an elongated body having first and second ends, a longitudinal axis, and top and bottom surfaces that extend along the longitudinal axis. A plurality of spaced anchors project from the top surface of the elongated body, each including a segment of an elongated channel extending therethrough that is aligned with the longitudinal axis of the elongated body. Each spaced anchor has an opening that is in communication with the segment of the elongated channel associated therewith. The spaced anchors define gaps therebetween that overlie the top surface of the elongated body. The elongated body is flexible and is more flexible at the locations of the gaps and less flexible at the locations of the spaced anchors. An elongated tissue closure device is secured to the anchors for applying pressure for sternal closure.

### BACKGROUND OF THE INVENTION

### Field of the Invention

The present patent application is generally related to medical devices and is more particularly related to systems, devices, and methods used for applying pressure for sternal closure after open chest surgery.

### Description of the Related Art

The sternum is a bone in the middle of the chest to which all of a patient's ribs are directly or indirectly attached. During open chest surgery (e.g., cardiac surgery), access is typically obtained by using a chest saw, or other cutting instrument to make a midline, longitudinal incision along the length of the patient's sternum to allow for lateral separation of the two opposing halves of the sternum. Typically, a sternal retractor is used to laterally separate the two opposing halves of the sternum.

At the conclusion of an open chest surgery, the sternum is typically closed using stainless steel wires that are placed through the sternum or around the sternum intercostal (i.e., sternal cerclage). In most instances, stainless steel wires are used for sternal cerclage because they are inexpensive and easy to use.

Many elderly patients undergoing open chest surgery (e.g., open heart surgery) suffer from many co-morbidities such as diabetes and osteoporosis, which may delay healing or make the patients susceptible to instability in the sternal fusion and delayed healing. When stainless steel wires are used to cerclage the sternum in patients with osteoporosis, the load placed on the sternum to approximate the bone edges may cause the relatively thin stainless steel wires to cut through the soft osteoporotic bone.

Thus, there remains a need for sternal closure solutions that allow for the continued use of wires for applying pressure, but which distribute the pressure load over a wider area for preventing the wires from cutting into the underlying bone. There also remains a need for pressure distribution implants that include elongated tissue closure devices that may be used for applying pressure for sternal closure, whereby the pressure distribution implants are located between the elongated tissue closure device and the underlying bone of the sternum.

### SUMMARY OF THE INVENTION

In one embodiment, a pressure distribution implant adapted for approximating opposing edges of bone during sternal closure preferably includes a pressure applying body (e.g., buttress material; an elongated body) that is located between an elongated tissue closure device (e.g., a wire; a suture; a polymer band) and the sternum to distribute the load from the elongated tissue closure device over a wider area for preventing the elongated tissue closure device from cutting into the bone.

In one embodiment, a pressure distribution implant may include a C-shaped structure (e.g., a normally straight flexible body may be flexed into the C-shaped structure) for simple cerclage through single intercostal spaces.

In one embodiment, a pressure distribution implant may include an X-shaped structure having a figure eight configuration for passing through adjacent intercostal spaces.

In one embodiment, a pressure distribution implant having an X-shape may be hinged and/or include a pivot point (e.g., a pivot pin) to allow for varying angles due to differences in intercostal spacing.

In one embodiment, an outer or top surface of a pressure distribution implant may have a softer segmented layer with a channel or groove to hold the elongated tissue closure device (e.g., a stainless steel wire) in place for preventing the elongated tissue closure device from sliding off the implant. In one embodiment the implant material is preferably flexible to conform the shape of the sternum to facilitate applying pressure to the sternum. In one embodiment, the implant has a harder inner or bottom surface for distributing the compressive and/or pressure load from the elongated tissue closure device over a larger surface area of the sternum.

In one embodiment, an elongated tissue closure device configured to apply pressure for sternal closure may be snap-fit or press fit into a groove, channel and/or anchor(s) of a body (e.g., an elongated body; a flexible body) of a pressure distribution implant.

In one embodiment, the ends of an elongated tissue closure device may be joined together and tightened (e.g., twisted) to apply pressure for bone reduction per current techniques.

In one embodiment, a pressure distribution implant for approximating opposing bone edges for sternal closure preferably includes an elongated body (e.g., a flexible body) having a first end, a second end, and a longitudinal axis that extends from the first end to the second end of the elongated body.

In one embodiment, the flexible body desirably has top and bottom surfaces that extend along the longitudinal axis. In one embodiment, the flexible body includes a biocompatible polymer material such as PEEK. In one embodiment, when the flexible body is used for sternal closure, the bottom surface of the flexible body preferably engages a sternum for applying pressure when approximating the opposing bone edges of the sternum.

In one embodiment, the pressure distribution implant desirably includes a plurality of anchors projecting from the top surface of the flexible body and being spaced from one another between the first and second ends of the flexible body. In one embodiment, each anchor may have a segment of an elongate channel extending therethrough that is aligned with the longitudinal axis of the flexible body. In one embodiment, each anchor has an opening that is in communication with the segment of the elongated channel associated therewith. An elongated tissue closure device, such as a wire, an elongated stainless steel wire, a suture, or a polymer band, may be passed through the openings for securing the elongated tissue closure device to the spaced anchors.

In one embodiment, the plurality of the anchors define gaps therebetween that overlie the top surface of the flexible body. The gaps are desirably located between the spaced anchors.

In one embodiment, the plurality of anchors are evenly spaced from one another between the first and second ends of the elongated body.

In one embodiment, the gaps located between the anchors are evenly spaced from one another between the first and second ends of the flexible body.

In one embodiment, due to the presence of the anchors that are spaced from one another, the flexible body in thicker and/or has more bulk at the location of the spaced anchors. In one embodiment, the flexible body is thinner and/or has less bulk at the locations of the gaps.

In one embodiment, the flexible body of the pressure distribution implant is more flexible at the location of the gaps and is less flexible at the location of the spaced anchors.

In one embodiment, each of the anchors has a constant length that extends along the longitudinal axis of the flexible body.

In one embodiment, the segments of the elongated channel define an elongated channel diameter, and each opening associated with one of the anchors has a width that is less than the diameter of the elongated channel.

In one embodiment, the segments of the elongated channel that pass through the respective anchors preferably define tube-shaped hollows that are aligned with the longitudinal axis of the flexible body.

In one embodiment, the openings of the anchors are located at the upper ends of the respective anchors. In one embodiment, the openings are aligned with the longitudinal axis of the flexible body.

In one embodiment, an elongated tissue closure device, such as an elongated wire, is disposed within the segments of the elongated channel of the respective anchors. In one embodiment, the elongated tissue closure device preferably extends through the anchors and along the longitudinal axis of the flexible body for being secured over the top surface of the flexible body.

In one embodiment, after the elongated tissue closure device has been secured to the anchors, the elongate tissue closure device may be used for wrapping the bottom surface of the flexible body around a sternum for approximating the opposing bone edges of the sternum during sternal closure. The ends of the elongated tissue closure device may be joined, twisted and/or tightened for applying pressure via the flexible body of the implant for approximating the opposing bone edges of the sternum. The flexible body preferably spaces the elongated tissue closure device from the bone and distributes the pressure forces from the elongated tissue closure device over the entire bottom surface of the flexible body of the implant. Thus, the flexible body distributes the pressure forces to the bone rather than the elongated tissue closure device being in direct contact with the bone, which prevents the elongate tissue closure device (e.g., a wire) from cutting into the bone of the sternum as pressure is applied to the sternum via the elongate tissue closure device.

In one embodiment, the elongated tissue closure device has a cross-sectional diameter that is greater than the width of the openings of the respective anchors. The anchors may have arms on either side of openings that flex away from one another for enabling the elongated tissue closure device to be pressed through the respective openings.

In one embodiment, the cross-sectional diameter of the elongated tissue closure device is greater than the inner diameter of the elongated channel. Thus, prior to tightening the elongated tissue closure device for applying pressure, the elongated tissue closure device may slide freely within the elongated channel and along the length of the elongated body for positioning the elongated tissue closure device along the length of the elongated body.

In one embodiment, the elongated tissue closure device may be made of a first material that is relatively softer and more resilient than a second material that covers the bottom surface of the flexible body. The relatively harder bottom surface of the pressure distribution implant preferably distributes the pressure over a wider surface area of the sternum.

In one embodiment, a pressure distribution implant for sternal closure desirably includes a flexible body having top and bottom surfaces that extend along a longitudinal axis of the flexible body, and a plurality of anchors projecting from the top surface of the flexible body, the anchors being spaced from one another along the longitudinal axis of the flexible body.

In one embodiment, the anchors define a series of gaps therebetween that are located between the spaced anchors. In one embodiment, the gaps preferably overlie the top surface of the flexible body and are in alignment with the longitudinal axis of the flexible body.

In one embodiment, each anchor has a segment of an elongated channel extending therethrough, the elongated channel being in alignment with the longitudinal axis of the flexible body.

In one embodiment, each anchor has an outer wall with an opening formed therein that is in communication with the respective segment of the elongated channel extending through the anchor.

In one embodiment, the openings of the respective anchors are aligned with the longitudinal axis of the flexible body.

In one embodiment, the segments of the elongated channel have cross-sectional diameters, each opening having a width that is less than the cross-sectional diameters of the respective segments of the elongated channel.

In one embodiment, an elongated tissue closure device may be secured over the top surface of the flexible body by the spaced anchors, the elongated tissue closure device extending through the segments of the elongated channel that pass through the respective anchors for being aligned with the longitudinal axis of the flexible body.

In one embodiment, the flexible body may be flexed/bent from a straight configuration to a curved configuration (e.g., a C-shaped configuration), such as when the flexible body is at least partially wrapped around a sternum for approximating the opposing edges of the bone of the sternum during sternal closure. In one embodiment, when the flexible body is in the curved configuration (e.g., the C-shaped configuration), the top surface of the flexible body defines a convex surface, and the bottom surface of the flexible body defines a concave surface that is adapted for being in direct contact with the sternum.

In one embodiment, a pressure distribution implant may include a second flexible body having top and bottom surfaces that extend along a second longitudinal axis that intersects with the first longitudinal axis of the first flexible body, and a plurality of second anchors projecting from the top surface of the second flexible body, the second anchors being spaced from one another along the second longitudinal axis of the second flexible body.

In one embodiment, the second anchors desirably define a series of second gaps therebetween that are located between the respective second anchors and that are in alignment with the second longitudinal axis of the second flexible body.

In one embodiment, each second anchor has a segment of a second elongated channel extending therethrough that is in alignment with the second longitudinal axis of the second flexible body.

In one embodiment, each second anchor has an outer wall with a second opening formed therein that is in communication with a segment of the second elongated channel passing through the second anchor associated therewith.

In one embodiment, an elongated tissue closure device (e.g., a wire) adapted to apply pressure for sternal closure may be secured over the top surface of the second flexible body by the second anchors. The elongated tissue closure device may extend through the respective segments of the second elongated channel and may be aligned with the second longitudinal axis of the second flexible body.

In one embodiment, the first and second flexible bodies cross one another and define an implant structure having an X-shaped configuration, which may facilitate conforming the implant to adjacent intercostal spaces of a sternum.

In one embodiment, a pressure distribution implant may include a pin that pivotally interconnects the first and second flexible bodies for enabling surgical personnel to selectively change the angle between the first longitudinal axis of the first flexible body and the second longitudinal axis of the second flexible body. The angle may be changed for customizing the shape of the pressure distribution implant to conform to the contour of an underlying sternum or adjacent intercostal spaces.

In one embodiment, a pressure distribution implant for sternal closure may include a flexible body having a first end, a second end, and a longitudinal axis that extends from the first end to the second end.

In one embodiment, the flexible body has top and bottom surfaces that extend along the longitudinal axis.

In one embodiment, a plurality of anchors project from the top surface of the flexible body and are spaced from one another between the first and second ends of the flexible body.

In one embodiment, each anchor may include a segment of an elongated channel that is aligned with the longitudinal axis of the flexible body, each anchor having an opening that is in communication with the segment of the elongated channel associated therewith.

In one embodiment, an elongated tissue closure device is secured over the top surface of the flexible body by the anchors, the elongated tissue closure device extending through the segments of the elongated channel that are aligned with the longitudinal axis of the flexible body.

In one embodiment, the elongated tissue closure device has a cross-sectional diameter that is greater than a width of the openings of the respective anchors.

In one embodiment, the spaced anchors define gaps therebetween that overlie the top surface of the flexible body and that are located between the spaced anchors.

In one embodiment, the flexible body is more flexible at the location of the gaps and is less flexible at the location of the spaced anchors.

In one embodiment, the flexible body may include a biocompatible polymer material such as PEEK.

In one embodiment, the anchors may be made of a first material that is softer and/or more flexible than a second material (e.g., a harder material; a metal band; a titanium band) that covers the bottom surface of the flexible body. In one embodiment, the harder bottom surface of the pressure distribution implant is configured to distribute the pressure forces from the elongated tissue closure device over a wider area of the sternum.

In one embodiment, the elongated tissue closure device may be a single wire or a double wire used for applying pressure through the flexible body of the pressure distribution implant.

In one embodiment, a first member (e.g., a first elongate body) of a pressure distribution implant has a recess formed in a top surface thereof, and a second member (e.g., a second elongated body) of the pressure distribution implant has a corresponding recess formed in a bottom surface thereof, whereby the first and second members are nested together and pivotally coupled within the respective recesses for enabling the first and second members to be pivoted to different angles relative to one another for customizing the shape of the pressure distribution implant to allow for varying angles due to differences in intercostal spacing.

In one embodiment, the first and second members of a pressure distribution implant may form a unitary X-shaped body, which is non-pivotable so that the angle between the first and second members will remain constant.

In one embodiment, a top surface of an elongated body of a pressure distribution implant may have a groove that is dimensioned to allow an elongated tissue closure device (e.g., a wire; a stainless steel wire; a suture) to be snap-fit or snug fit within the groove. The groove may be intermittent along the length of the elongated body of the pressure distribution implant to define groove segments that are spaced from one another. The groove segments may have respective lengths that extend along a longitudinal axis of the elongated body of the pressure distribution implant.

These and other preferred embodiments of the present patent application will be described in more detail herein.

### BRIEF DESCRIPTION OF THE DRAWINGS

FIG. 1 is a top view of a pressure distribution implant used for sternal closure, the pressure distribution implant having a plurality of anchors spaced from one another along a length thereof, in accordance with one embodiment of the present patent application.

FIG. 2 is a cross-sectional view of the pressure distribution implant shown in FIG. 1 taken along line 2-2 thereof including an anchor projecting above a top surface of the pressure distribution implant, in accordance with one embodiment of the present patent application.

FIG. 3 is a cross-sectional view off the pressure distribution implant shown in FIG. 1 taken along line 3-3 thereof including a gap located between two adjacent anchors, in accordance with one embodiment of the present patent application.

FIG. 4A shows a stage of a method of securing an elongated tissue closure device to an anchor of a pressure distribution implant, in accordance with one embodiment of the present patent application.

FIG. 4B shows another stage of a method of securing the elongated tissue closure device of FIG. 4A to the wire anchor of the pressure distribution implant, in accordance with one embodiment of the present patent application.

FIG. 5 shows the pressure distribution implant of FIG. 1 after an elongated tissue closure device has been secured to the spaced anchors for affixing the elongated tissue closure device over a top surface of the implant, in accordance with one embodiment of the present patent application.

FIG. 6 is a perspective top view of a pressure distribution implant having a curved configuration for applying pressure for sternal closure, the pressure distribution implant including a plurality of spaced anchors and gaps located between adjacent anchors, in accordance with one embodiment of the present patent application.

FIG. 7 shows a perspective bottom view of the pressure distribution implant shown in FIG. 6.

FIG. 8 shows the pressure distribution implant of FIG. 6 after an elongated tissue closure device has been secured to the spaced anchors, in accordance with one embodiment of the present patent application.

FIG. 9 shows a perspective bottom view of the pressure distribution implant shown in FIG. 8.

FIG. 10 is a top view of a pressure distribution implant including first and second elongated bodies that cross one another to define an X-shaped structure, in accordance with one embodiment of the present patent application.

FIG. 11 shows the pressure distribution implant of FIG. 10 after a first segment of an elongated tissue closure device has been secured to spaced anchors of the first elongated body and a second segment of the elongated tissue closure device has been secured to spaced anchors of the second elongated body, in accordance with one embodiment of the present patent application.

FIG. 12 is a perspective top view of a pressure distribution implant having first and second elongated bodies that cross one another to define an X-shaped structure, the first and second elongated bodies being pivotally interconnected for selectively changing the angle between the first and second elongated bodies, in accordance with one embodiment of the present patent application.

FIG. 13 shows a magnified view of a pivotable joint including a pivot pin that pivotally interconnects the first and second elongated bodies of the pressure distribution implant shown in FIG. 12.

FIG. 14 shows the pressure distribution implant of FIG. 12 after an elongated tissue closure device has been secured to spaced anchors that project from a top surface of the elongated body of the implant, in accordance with one embodiment of the present patent application.

FIG. 15A is a perspective view of a distal end of a pressure distribution implant having soft and flexible anchors that project from a top surface of the implant and a bottom surface that includes a material that is harder and less flexible than the material used to make the anchors, in accordance with one embodiment of the present patent application.

FIG. 15B is a perspective side view of the distal end of the pressure distribution implant shown in FIG. 15A.

FIG. 16 is a top view of a pressure distribution implant used for sternal closure, the pressure distribution implant including a sternal plate having a top surface and anchors projecting from the top surface of the sternal plate, in accordance with one embodiment of the present patent application.

FIG. 17 shows the pressure distribution implant of FIG. 16 after an elongated tissue closure device has been secured to the anchors overlying the top surface of the sternal plate of the pressure distribution implant, in accordance with one embodiment of the present patent application.

FIG. 18 is a top view of the pressure distribution implant shown in FIG. 17, the pressure distribution implant applying pressure to a sternum for sternal closure.

FIG. 19A is a perspective view of a pressure distribution implant including an elongated body having a first end with a first tapered surface, a second end with a second tapered surface, an elongated channel extending along the length of the elongated body, and an opening that provides access to the elongated channel, in accordance with one embodiment of the present patent application.

FIG. 19B shows the pressure distribution implant of FIG. 19A and an elongated tissue closure device inserted into the elongated channel of the pressure distribution implant.

FIG. 20 shows a system for performing sternal closure including a first pressure distribution implant wrapped around a first side of a sternum, a second pressure distribution implant wrapped around a second side of the sternum, and an elongated tissue closure device inserted into elongated channels of the respective first and second pressure distribution implants for effectuating sternal closure, in accordance with one embodiment of the present patent application.

FIG. 21 shows a system for performing sternal closure including a first pressure distribution implant wrapped around a first side of a sternum, a second pressure distribution implant wrapped around a second side of the sternum, and an elongated tissue closure device inserted into elongated channels of the respective first and second pressure distribution implants for effectuating sternal closure, in accordance with one embodiment of the present patent application.

### DETAILED DESCRIPTION OF PREFERRED EMBODIMENTS

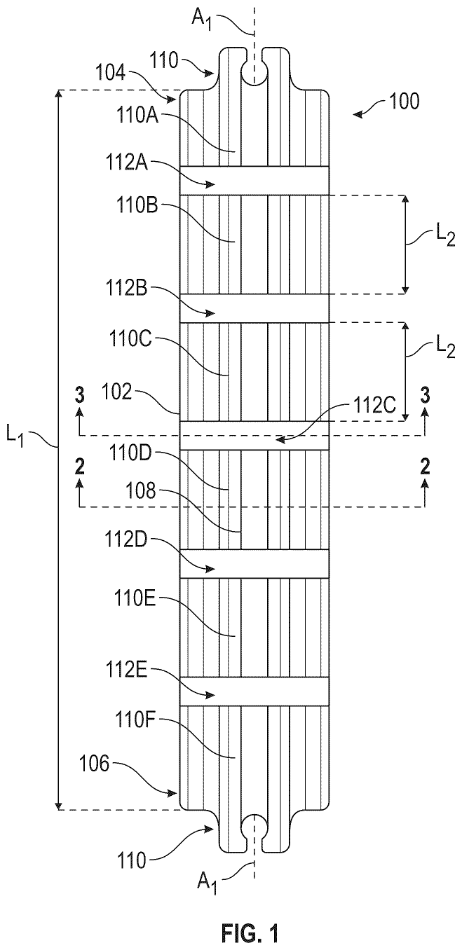

Referring to FIG. 1, in one embodiment, a pressure distribution implant 100 used to apply pressure for approximating the opposing edges of a sternum following open chest surgery preferably includes an elongated body 102 (e.g., a flexible body) having a first end 104 and a second end 106. In one embodiment, the elongated body 102 of the pressure distribution implant 100 may have a length L₁ that extends along a longitudinal axis A₁. In one embodiment, the elongated body 102 may be flexible (e.g., the ends of the elongated body may extend beyond the sternum and may be flexed/bent) for conforming to the shape of the sternum to facilitate applying pressure to the sternum for sternal closure. Thus, the elongated body may be transformed from a straight configuration to a curved or C-shaped configuration, whereby the ends of the implant may be at least partially wrapped around a sternum. In one embodiment, the elongated body may be made of a biocompatible polymer material such as PEEK.

In one embodiment, the pressure distribution implant 100 preferably includes an elongated channel 108 that extends over a top surface of the elongated body 102. The elongated channel 108 preferably extends between the first and second ends 104, 106 of the elongated body 102. In one embodiment, the elongated channel 108 preferably extends along the longitudinal axis A₁ of the elongated body 102. In one embodiment, the elongated channel 108 may be intermittent along the length of the elongated body. In one embodiment, an elongated tissue closure device (e.g., a wire; a suture) may be secured within the elongated channel 108.

In one embodiment, the pressure distribution implant 100 may include one or more snap-fit connectors (e.g., anchors) for securing an elongated tissue closure device over the top surface of the elongated body 102 for maintaining the elongated tissue closure device in contact with the implant as pressure is applied by tightening the elongated tissue closure device. In one embodiment, the elongated tissue closure device may have a cross-sectional dimension that is slightly smaller than the cross-sectional dimension of the elongated channel for enabling the elongated tissue closure device to slide freely within the elongated channel, such as when the position of the elongated tissue closure device is being adjusted relative to the elongated body of the implant.

In one embodiment, the pressure distribution implant 100 preferably includes a plurality of anchors 110A-110F that are spaced from one another over the top surface of the elongated body 102, which are configured for securing an elongated tissue closure device (e.g., a stainless steel wire) over the top surface of the elongated body. In one embodiment, the spaced anchors 110A-110F desirably project above the top surface of the elongated body 102. In one embodiment, each of the spaced anchors 110 preferably includes a segment of the elongated channel 108, whereby the elongated channel segments pass through the respective anchors along the length of the elongated body 102.

In one embodiment, the anchors 110A-110F are preferably integrally formed with the elongated body 102 and project above the top surface 114 (FIG. 3) of the elongated body 102. In one embodiment, the anchors 110A-110F are spaced from one another along the length of the elongated body. In one embodiment, each of the anchors 110A-110F preferably has a constant length L₂. In one embodiment, the pressure distribution implant 100 preferably includes a series of gaps 112A-112E, whereby each gap is located between two of the spaced anchors 110A-110F. In one embodiment, a single anchor 110 is bounded by two of the gaps 112, and a single gap 112 is bounded by two of the anchors 110.

In one embodiment, a pressure distribution implant may have an anchor that extends continuously along the length of an elongated body. In this embodiment, the pressure distribution implant does not have gaps (i.e., the gaps 112A-112E shown in FIG. 1), and the pressure distribution implant is made of a material that enables the elongated body to be bendable/flexible without the gaps.

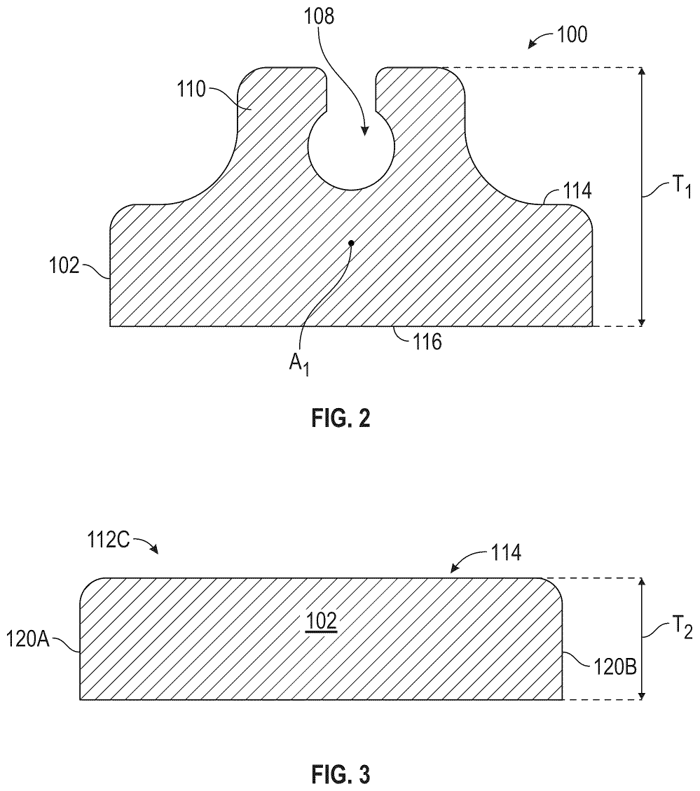

Referring to FIG. 2, in one embodiment, the elongated body 102 of the pressure distribution implant 100 preferably includes the top surface 114 and a bottom surface 116. When used for sternal closure, the bottom surface 116 preferably engages bone. The top and bottom surfaces 114, 116 preferably extend along the length of the elongated body 102. The pressure distribution implant 100 preferably includes the plurality of anchors 110A-110F (FIG. 1) that project above the top surface 114 of the elongated body 102. Each anchor 110 preferably defines a segment of the elongated channel 108 that extends along the longitudinal axis A₁ (FIG. 1) of the elongated body 102. In one embodiment, the elongated channel 108 may be intermittent along the length of the elongated body 102 and may only be located at the locations of the spaced anchors 110.

Referring to FIG. 3, in one embodiment, the gaps 112A-112E (FIG. 1) of the elongated body 102 are devoid of the anchors 110A-110F. FIG. 3 shows a section of the top surface 114 of the elongated body that defines a gap 112C, which is devoid of one of the anchors 110. At the location of the gap 112C, the top surface 114 extends across the width of the elongated body 102 and may be configured for supporting an elongated tissue closure device (e.g., an elongated, stainless steel wire) that extends along the top surface 114 of the elongated body 102.

In one embodiment, the elongated body 102 is flexible so that the ends of the elongated body may be bent, flexed and/or curved for conforming to the outer surface of a sternum for effectuating sternal closure. In one embodiment, the elongated body is made of a flexible material such as a biocompatible polymer (e.g., PEEK).

Referring to FIGS. 2 and 3, in one embodiment, the pressure distribution implant 100 preferably has a first thickness T₁ at the locations of the respective anchors 110A-110F (FIG. 1) and a second thickness T₂ at the locations of the respective gaps 112, whereby T₁ is greater than T₂. Due to the varying thickness at the locations of the anchors and the locations of the gaps, the elongated body of the pressure distribution implant may be more flexible and/or bendable at the locations of the gaps and less flexible and/or bendable at the locations of the anchors.

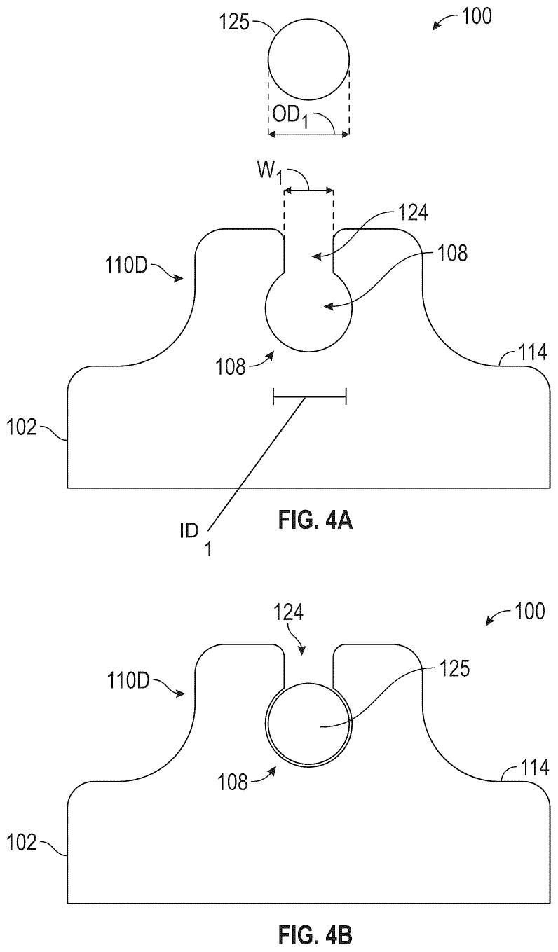

Referring to FIG. 4A, in one embodiment, an elongated tissue closure device 125 (e.g., a wire such as a stainless steel wire) may be anchored and/or secured over the top surface 114 of the elongated body 102 of the pressure distribution implant 100. In FIG. 4A, the anchor 110D projects above the top surface 114 of the elongated body 102. The anchor 110D desirably defines a segment of the elongated channel 108 that extends along the length of the elongated body 102.

In one embodiment, the elongated channel 108 may have a cross-sectional shape that defines a circle. In other embodiments, the cross-section of the elongated channel may define a different geometric shape (e.g., square, rectangular, triangular, polygon), such as a geometric shape that accommodates the cross-sectional shape of the elongated tissue closure device. For example, the elongated channel may have the square-shaped cross-section that generally matches the square-shaped cross-section of the elongated tissue closure device.

In one embodiment, the anchor 110D preferably includes an opening 124 formed therein through which the elongated tissue closure device 125 may be passed for anchoring and/or securing the elongated tissue closure device to the anchor 110D. In one embodiment, the opening 124 of the anchor 110D desirably has a width W₁ that is less than the outer diameter OD₁ of the elongated tissue closure device 125. After the elongated tissue closure device 125 has been pressed through the opening 124, the reduced width of the opening will hold the elongated tissue closure device 125 within the segment of the elongated channel 108 that passes through the anchor 110D.

In one embodiment, the anchor 110D is preferably made of a resilient material that enables the larger diameter elongated tissue closure device to be passed through the relatively smaller opening 124. In one embodiment, the size of the opening 124 will expand when the elongated tissue closure device 125 is pressed through the opening 124, and the opening will return to its original width W₁ when the elongated tissue closure device has been seated within the segment of the elongated channel 108 that passes through the anchor 110D. In one embodiment, after the elongated tissue closure device 125 has been forced/pressed through the opening 124, a section of the anchor 110D adjacent the opening 124 may engage and/or retain the elongated tissue closure device within the segment of the elongated channel 108 that passes through the anchor 110D.

Referring to FIGS. 4A and 4B, in one embodiment, the elongated tissue closure device 125 may be pressed and/or forced through the opening 124 of the anchor 110D for securing the elongated tissue closure device 125 within the segment of the elongated channel 108 that extends along the length of the elongated body 102. In one embodiment, a similar process is followed for securing the elongated tissue closure device 125 to each of the other anchors 110A-110F (FIG. 1) that are spaced from one another along the top surface 114 of the elongated body 102 of the pressure distribution implant 100.

Referring to FIGS. 4A and 4B, in one embodiment, the elongated tissue closure device 125 preferably has an outer diameter OD₁ that is slightly smaller than the inner diameter ID₁ (FIG. 4A) of the elongated channel 108 for enabling the elongated tissue closure device to slide freely within the elongated channel 108 for adjusting the position of the elongated tissue closing device 125 relative to the elongated body before the elongated tissue control device is tightened for applying pressure to the underlying bone for sternal closure.

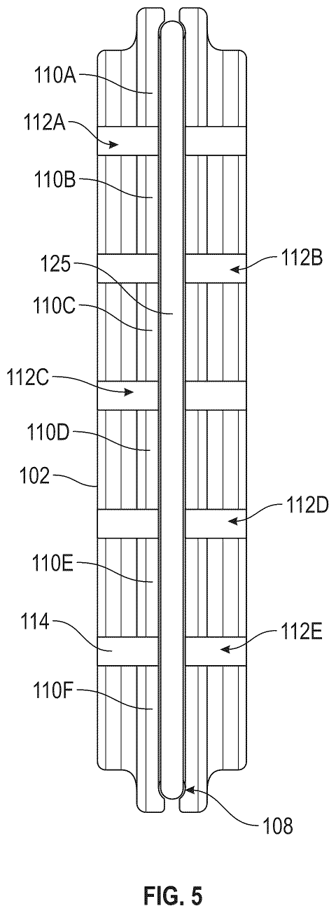

Referring to FIG. 5, in one embodiment, the elongated tissue closure device 125 is preferably secured over the top surface 114 of the elongated body 102 by pressing the elongated tissue closure device 125 through the series of openings 124 (FIG. 4A) that are present in the respective anchors 110A-110F. In one embodiment, the elongated tissue closure device 125 preferably extends along the elongated channel 108 that is defined by each of the anchors 110A-110F. The elongated tissue closure device 125 desirably extends through the gaps 112A-112E that are located between the respective spaced anchors 110A-110F.

In one embodiment, the elongated body 102 of the pressure distribution implant 100 is more flexible at the location of the gaps 112A-112E and is less flexible at the location of the anchors 110A-110F.

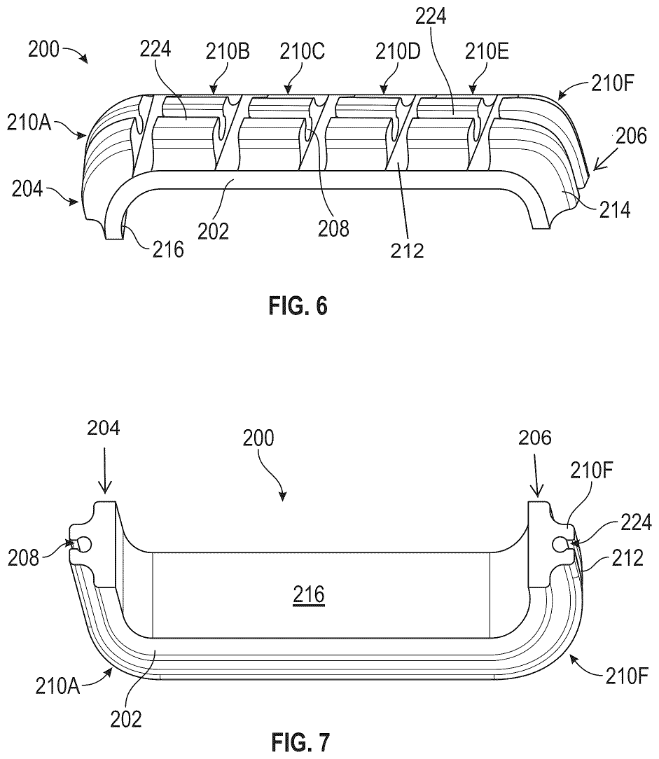

Referring to FIGS. 6 and 7, in one embodiment, an elongated body 202 of a pressure distribution implant 200 may be made of a flexible material (e.g., a biocompatible polymer material such as PEEK) so that first and second ends 204, 206 of the elongated body may be bent and/or flexed into a curved configuration (e.g., a C-shaped configuration) for conforming to the outer surface of the sternum during a sternal closure procedure. In one embodiment, the elongated body 202 preferably includes the first end 204, the second end 206 and an elongated channel 208 that extends along the length of the elongated body between the first and second ends 204, 206 thereof.

In one embodiment, the elongated body 202 preferably includes a plurality of anchors 210A-210F that are spaced from one another along the length of the elongated body 202. In one embodiment, each respective anchor 210A-210F preferably includes an opening 224 that enables an elongated tissue closure device to be pressed and/or forced through the respective openings 224 for being secured by the spaced anchors 210A-210F. In one embodiment, after the elongated tissue closure device has been anchored and/or secured over a top surface 214 of the elongated body 202, the elongated tissue closure device preferably extends along the elongated channel 208 and through each of the respective anchors 210A-210F.

In one embodiment, the elongated body 202 has the top surface 214 and a bottom surface 216. The bottom surface 216 is preferably adapted for engaging the bone of a sternum when the elongated body 202 of the pressure distribution implant 200 is at least partially wrapped around the sternum during a sternal closure procedure.

Referring to FIG. 6, in one embodiment, the pressure distribution implant 200 preferably has gaps 212 that are located between adjacent anchors (e.g., anchors 210D and 210E). In one embodiment, the laterally extending surface that defines the gap 212 is below the lower end of the elongated channel 208. In one embodiment, the laterally extending surface that defines the gap 212 may be relatively higher is relation to the lower end of the elongated channel 208. For example, the surface that defines the gap 212 may be flush with the lower end of the elongated channel.

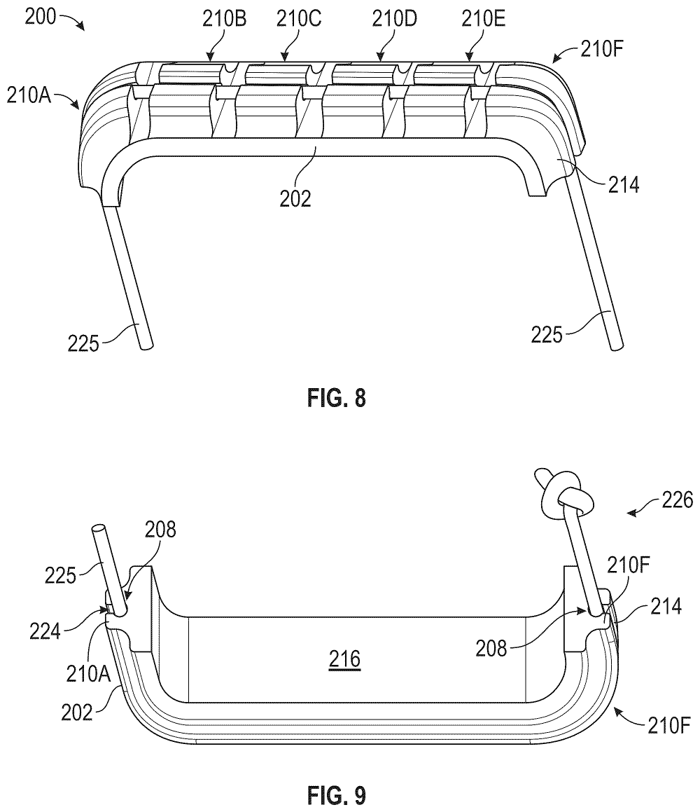

Referring to FIGS. 8 and 9, in one embodiment, an elongated tissue closure device 225 may be pressed through the respective openings 224 (FIG. 6) of the spaced anchors 210A-210F for anchoring and/or securing the elongated tissue closure device within the segments of the elongated channel 208 (FIG. 6) that extend along the length of the elongated body 202 of the pressure distribution implant 200.

Referring to FIG. 9, in one embodiment, the free ends of the elongated tissue closure device 225 may be joined together via one or more twists 226 for tightening the elongated tissue closure device so that the bottom surface 216 of the elongated body 202 may be utilized for applying pressure to the sternum to approximate the opposing edges of the bone of the sternum during a sternal closure procedure. The bottom surface 216 of the elongated body 202 provides a pressure applying surface that is significantly broader than the outer diameter OD₁ (FIG. 4A) of the elongated tissue closure device to preferably distribute the pressure forces over a broader surface area of the sternum, while also preventing the elongated tissue closure device from cutting into the bone of the sternum.

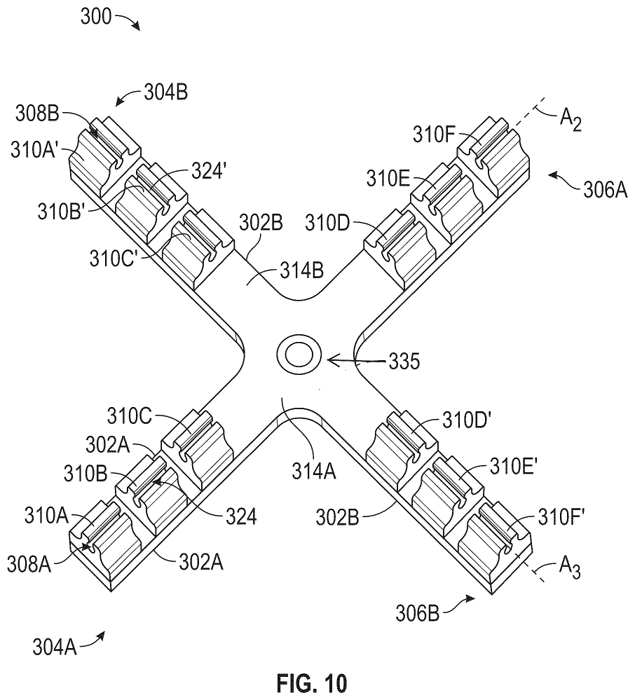

Referring to FIG. 10, in one embodiment, a pressure distribution implant 300 preferably includes a first elongated body 302A having a first end 304A and a second end 306A. The pressure distribution implant 300 preferably includes a second elongated body 302B having a first end 304B and second end 306B. In one embodiment, the first and second elongated bodies extend along respective axes A₂, A₃ that intersect one another at a junction 335. In one embodiment, the first and second elongated bodies 302A, 302B may intersect one another at an angle of about 90 degrees so that the pressure distribution implant 300 has an X-shaped configuration. In one embodiment, the first and second elongated bodies 302A, 302B may be made of a flexible material (e.g., a biocompatible polymer such as PEEK) so that the ends of the first and second elongated bodies 302A, 302B of the pressure distribution implant 300 may be at least partially wrapped around the sternum when applying pressure for approximating the opposing edges of the sternum during sternal closure.

In one embodiment, the first elongated member 302A has a plurality of first anchors 310A-310F that are spaced from one another along the length of the first elongated member. The spaced first anchors 310A-310F preferably project above a top surface 314A of the first elongated body 302A. In one embodiment, the first anchors 310A-310F have respective heights that are no greater than ½ of the total height of the pressure distribution implant 300. In one embodiment, the first anchors 310A-310F define a first elongated channel 308A that extends along the length of the first elongated body 302A. As will be described in more detail below, in one embodiment, an elongated tissue closure device (e.g., a wire) may be pressed and/or forced through the respective openings 324 of the spaced first anchors 310A-310F for securing an elongated tissue closure device over the top surface 314A of the first elongated body 302A, whereupon the secured elongated tissue closure device extends along the first elongated channel 308A.

In one embodiment, the second elongated body 302B preferably includes a plurality of second anchors 310A′-310F′ that are spaced from one another between the first end 304B and the second end 306B of the second elongated body 302B. In one embodiment, the second anchors 310A′-310F′ have respective heights that are no greater than % of the total height of the pressure distribution implant 300. In one embodiment, each of the spaced second anchors 310A′-310F′ preferably defines an opening 324′ through which an elongated tissue closure device may be pressed to secure the elongated tissue closure device within a second elongated channel 308B defined by each of the second spaced anchors 310A′-310F′. In one embodiment, the second elongated channel 308B extends over the top surface 314B of the second elongated member 302B so that the elongated tissue closure device may be secured over the top surface 314B of the second elongated body 302B.

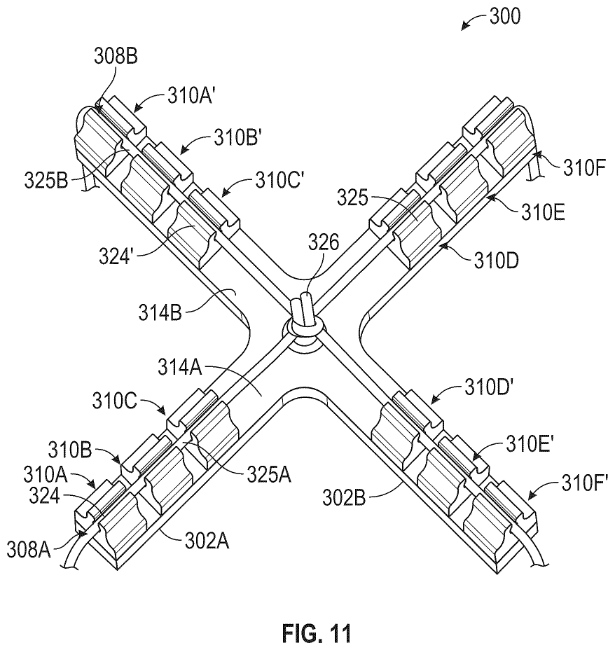

Referring to FIG. 11, in one embodiment, a first segment 325A of an elongated tissue closure device 325 (e.g., a stainless steel wire; a suture) may be secured to the plurality of spaced anchors 310A-310F that are spaced along the top surface 314A of the first elongated body 302A. The first segment 325A of the elongated tissue closure device 325 is preferably aligned with the first elongated channel 308A (FIG. 10) that is in communication with the openings 324 of each of the first anchors 310A-310F.

In one embodiment, a second section 325B of the elongated tissue closure device 325 may be secured to the second anchors 310A′-310F′ that extend over the top surface 314B of the second elongated body 302B of the pressure distribution implant 300. In one embodiment, the second section 325B of the elongated tissue closure device 325 is pressed through the openings 324′ of the second spaced anchors 310A′-310F′. After the second section 325B of the elongated tissue closure device 325 is secured to the second anchors 310A′-310F′, the second section 325B is preferably positioned within the second elongated channel 308B that is defined by the second anchors 310A′-310F′ of the second elongated body 302B of the pressure distribution implant 300. In one embodiment, the free ends of the elongated tissue closure device 325 may be joined together and connected with a twist 326 for tightening the elongated tissue closure device 325 and applying pressure to the sternum via the first and second elongated bodies 302A, 302B for approximating opposing edges of a sternum during a sternal closure procedure.

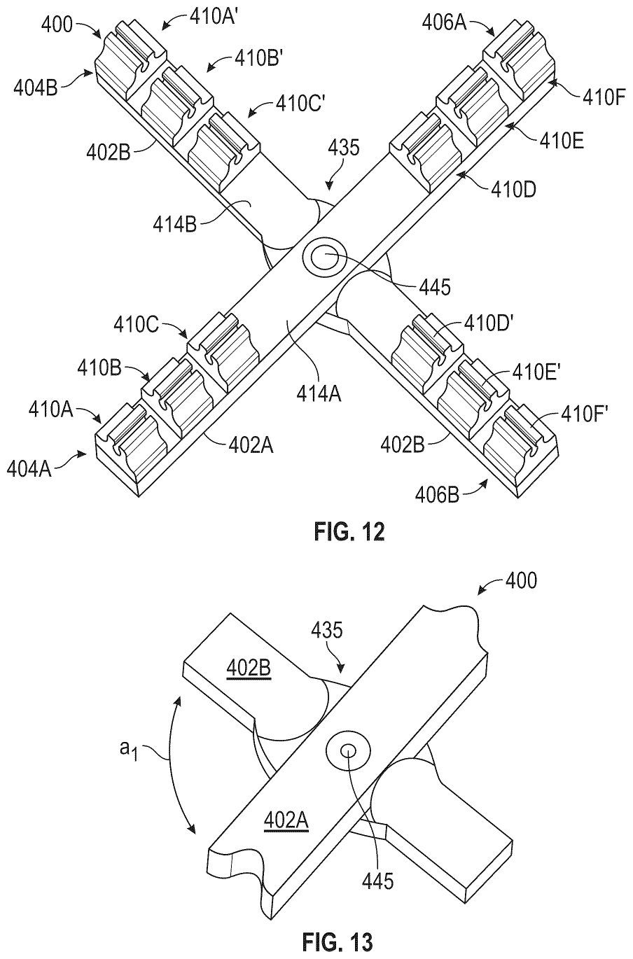

Referring to FIG. 12, in one embodiment, a pressure distribution implant 400 preferably has a generally X-shaped configuration including a first elongated body 402A that intersects with a second elongated body 402B. The first elongated body 402A preferably has a series of first anchors 410A-410F that are spaced from one another between a first end 404A and a second end 406A of the first elongated body 402A. As described herein, the spaced first anchors 410A-410F may be utilized for securing an elongated tissue closure device (e.g., a wire; a polymer band) over a top surface 414A of the first elongated member 402A. In one embodiment, the first anchors 410A-410F have respective heights that are no greater than ½ of the total height of the pressure distribution implant 400.

In one embodiment, the pressure distribution implant 400 preferably includes a second elongated member 402B having a plurality of second anchors 410A′-410F′ that are spaced from one another between the first end 404B and the second end 406B of the second elongated member 402B. As described herein, second anchors 410A′-410F′ of the second elongated body 402B may be utilized for securing an elongated tissue closure device (e.g., a wire) over the top surface 414B of the second elongated member 402B. In one embodiment, the second anchors 410A′-410F′ have respective heights that are no greater than % of the total height of the pressure distribution implant 400.

Referring to FIGS. 12 and 13, in one embodiment, the first and second elongated members 402A, 402B of the pressure distribution implant 400 are preferably joined together at a pivotable junction 435 having a pivot pin 445 that pivotally couples the first and second elongated bodies 402A, 402B together so that the X-shaped configuration of the pressure distribution implant 400 may be modified. As shown in FIG. 13, the pivot pin 445 at the pivotable junction 435 enables the angle α₁ between the first and second elongated bodies 402A, 402B to be adjusted and/or modified so that the pressure distribution implant 400 may conform to the shape of a sternum due to differences in intercostal spaces.

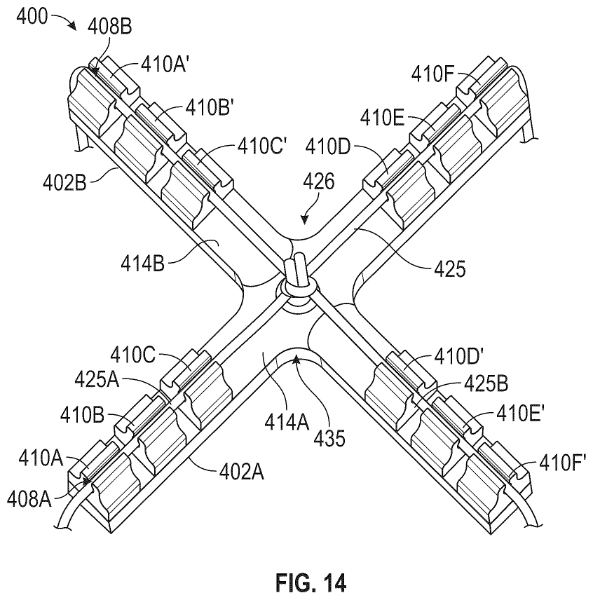

Referring to FIG. 14, in one embodiment, an elongated tissue closure device 425 may be secured over the top surfaces 414A, 414B of the respective first and second elongated bodies 402A, 402B of the pressure distribution implant 400. In one embodiment, a first segment 425A of the elongated tissue closure device 425 is secured over the top surface 414A of the first elongated body 402A using the spaced anchors 410A-410F. The first section 425A of the elongated tissue closure device 425 is preferably positioned within the elongated channel 408A that extends through the respective anchors 410A-410F.

In one embodiment, a second section 425B of the elongated tissue closure device 425 is preferably secured over the top surface 414B of the second elongated body 402B using the second anchors 410A′-410F′ that are spaced from one another along the length of the second elongated body 402B.

In one embodiment, the first and second sections 425A, 425B of the elongated tissue closure device 425 may cross one another at the pivotable junction 435 of the first and second elongated bodies 402A, 402B. In one embodiment, the free ends of the elongated tissue closure device 425 may be joined together in a twist 426 for tightening the elongated tissue closure device and applying pressure for approximating opposing edges of a sternum during sternal closure. In one embodiment, the respective ends of the first and second elongated bodies are flexible and may be bent and/or flexed into a figure eight configuration for conforming to adjacent intercostal spaces. In one embodiment, the ends of the elongated bodies extend beyond the sternum and may be flexed/bent for being at least partially wrapped around the sternum for performing a sternal closure procedure. An elongated tissue closure device (e.g., a wire) secured to the first and second elongated bodies may be tightened for applying pressure to the sternum via the pressure distribution implant.

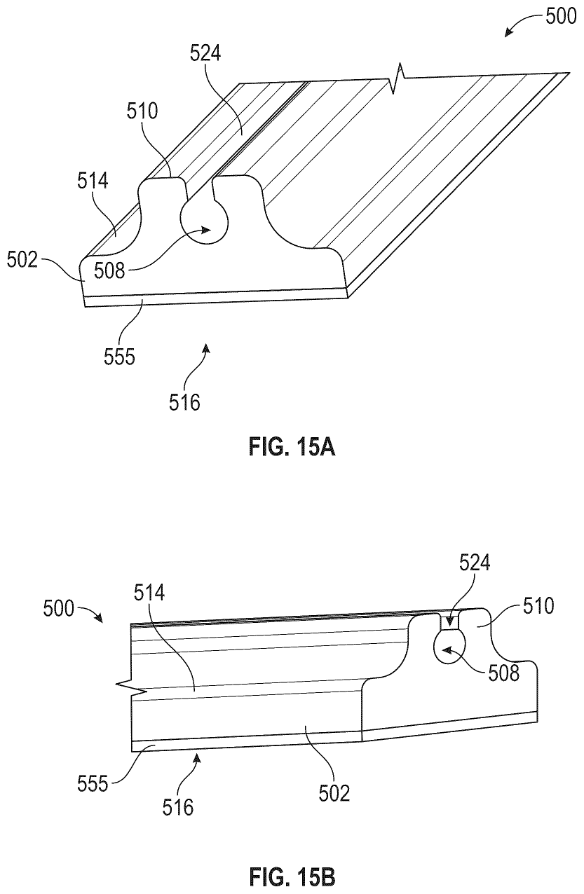

Referring to FIGS. 15A and 15B, in one embodiment, a pressure distribution implant 500 preferably includes an elongated body 502 (e.g., similar to the elongated body 102 shown in FIG. 1) having a top surface 514 and a bottom surface 516. In one embodiment, the pressure distribution implant 500 preferably includes one or more anchors 510 that projects above the top surface 514 of the elongated body 502. The one or more anchors 510 preferably define an elongated channel 508 that extends along the length of the elongated body 502. Each anchor 510 preferably has an opening 524 that is configured for enabling an elongated tissue closure device to be pressed therethrough for being secured by the respective anchors and within the elongated channel 508 that extends through the anchor 510.

In one embodiment, the anchors 510 of the elongated body are made of a relatively softer and/or more flexible material and the bottom surface 516 of the elongated body is covered and/or comprises a relatively harder and/or less flexible material than is used to make the anchors. In one embodiment, the bottom surface 516 of the elongated body 502 is covered by a band 555 (e.g., a metal band; a titanium band) that provides a relatively harder surface that extends over the bottom surface 516 of the elongated body 502 for more effectively distributing pressure to the bone of the sternum during a sternal closure procedure.

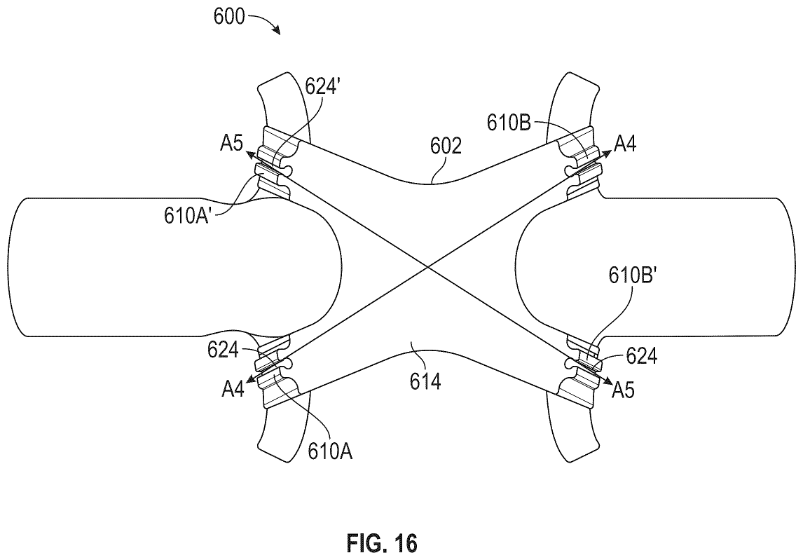

Referring to FIG. 16, in one embodiment, a pressure distribution implant 600 preferably includes a sternal plate 602 (e.g., a body made of a biocompatible polymer such as PEEK) having a top surface 614 and a bottom surface (not shown) that contacts the sternum. In one embodiment, the sternal plate 602 preferably includes a first pair of anchors 610A, 610B that are spaced from one another and that are aligned with a first axis A₄. Each anchor 610A, 610B has an opening 624 formed therein that is configured for enabling an elongated tissue closure device to be pressed therethrough for being secured by the first pair of anchors 610A, 610B over the top surface 614 of the plate 602. The free ends of the sternal plate adjacent the anchors may be made of a flexible material for being flexed/bent around the sternum during a sternal closure procedure. In FIG. 16, the free ends of the sternal plate 602 are bent around the sternum and are not visible in the drawing figure.

In one embodiment, the pressure distribution implant 600 preferably includes a second pair of anchors 610A′, 610B′ that are aligned along a second axis A₅ that intersects the first axis A₄. Each of the second pair of anchors 610A′, 610B′ preferably has an opening 624′ through which an elongated tissue closure device may be pressed for securing the elongated tissue closure device over the top surface 614 of the plate 602.

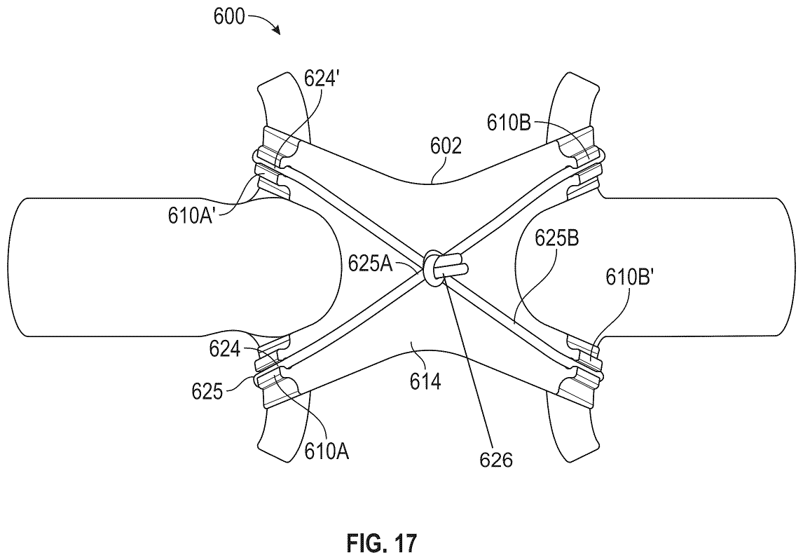

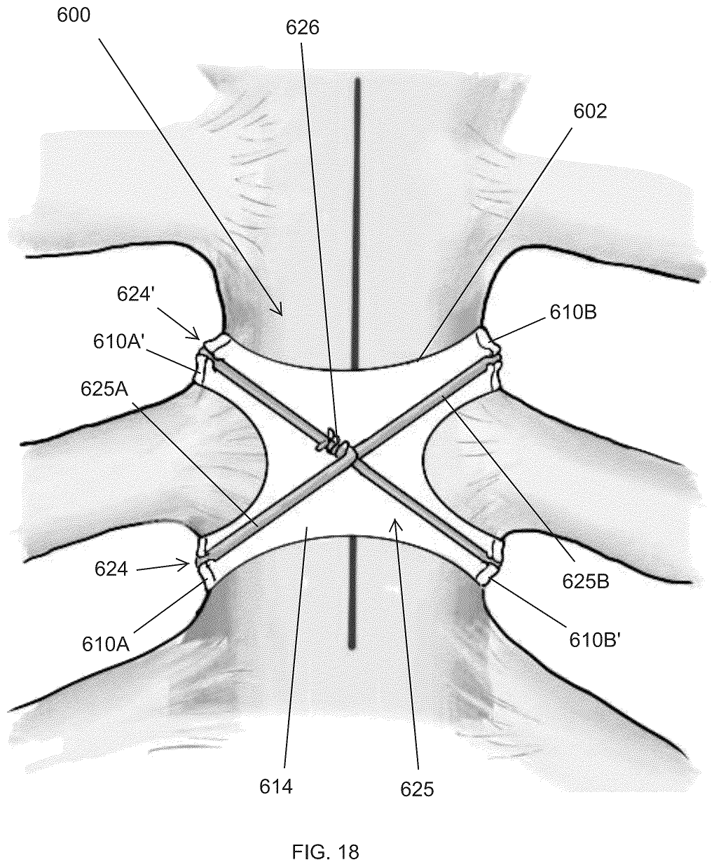

Referring to FIGS. 17 and 18, in one embodiment, a first section 625A of an elongated tissue closure device 625 may be secured over the top surface 614 of the plate 602 of the pressure distribution implant 600 by pressing the first section 625A of the elongated tissue closure device through the openings 624 of the first pair of anchors 610A, 610B. In one embodiment, the elongated tissue closure device may have a figure eight configuration whereby a second section 625B of the elongated tissue closure device is secured over the top surface 614 of the plate 602 by pressing the second section 625B through the openings 624′ of the second pair of anchors 610A′, 610B′. The ends of the elongated tissue closure device 625 may be joined together around the sternum and may be tightened via a twist 626 (e.g., a knot) for applying pressure to the sternum to effectuate sternal closure.

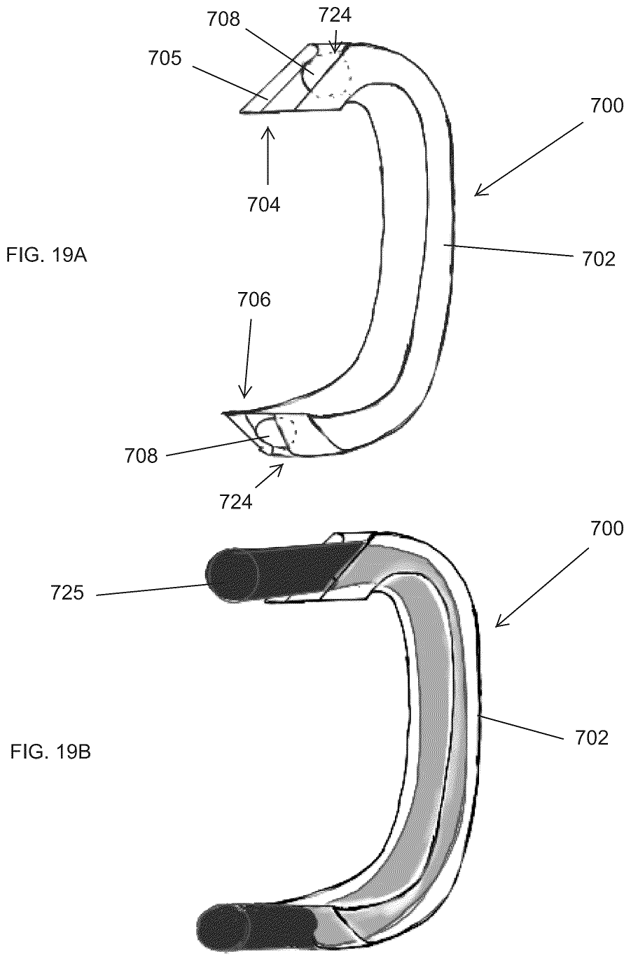

Referring to FIG. 19A, in one embodiment, a pressure distribution implant 700 used to apply pressure for approximating the opposing edges of a sternum following open chest surgery preferably includes an elongated body 702 (e.g., a flexible body) having a first end 704 with a first tapered surface 705 and a second end 706 with a second tapered surface 707. In one embodiment, the elongated body 702 may normally have a straight and/or flat configuration that may be flexed/bent into a curved configuration for engaging the bone of a sternum. In one embodiment, the elongated body 702 may be flexible for conforming to the shape of the underlying sternum to facilitate applying pressure to the sternum for sternal closure. In one embodiment, the elongated body may be made of a biocompatible polymer material such as PEEK.

In one embodiment, the elongated body 702 may be made of a flexible or bendable material. In one embodiment, the elongated body 702 may normally have a straight configuration, whereby the first and second ends 704, 706 of the elongated body 702 may be bent/flexed over the sternum when an elongated tissue closure device (e.g., a wire) is tightened for performing a sternal closure operation. In one embodiment, the elongated body 702 preferably has a length that is sufficient to enable the first and second ends 704, 706 of the elongated body to extend beyond the sternum at each end to allow the first and second ends to be bent/flexed over the sternum.

In one embodiment, the pressure distribution implant 700 preferably includes an elongated channel 708 that extends along the length of the elongated body 702. The elongated channel 708 preferably extends between the first and second ends 704, 706 of the elongated body 702. In one embodiment, the elongated channel 708 may be intermittent along the length of the elongated body.

In one embodiment, the pressure distribution implant 700 preferably includes an elongated opening 724 that is in communication with the elongated channel for enabling an elongated tissue closure device to be pressed through the elongated opening 724 for being seated within the elongated channel 708.

Referring to FIG. 19B, in one embodiment, an elongated tissue closure device 725 (e.g., a wire) may be secured within the elongated channel 708 (FIG. 19A) of the elongated body 702 of the pressure distribution implant 700. In one embodiment, the elongated channel 708 preferably has a cross-sectional diameter that is slightly larger than the cross-sectional diameter of the elongated tissue closure device 725 (e.g., a wire) to enable the elongated tissue closure device 725 to slide freely within the elongated channel 708 of the pressure distribution implant 700.

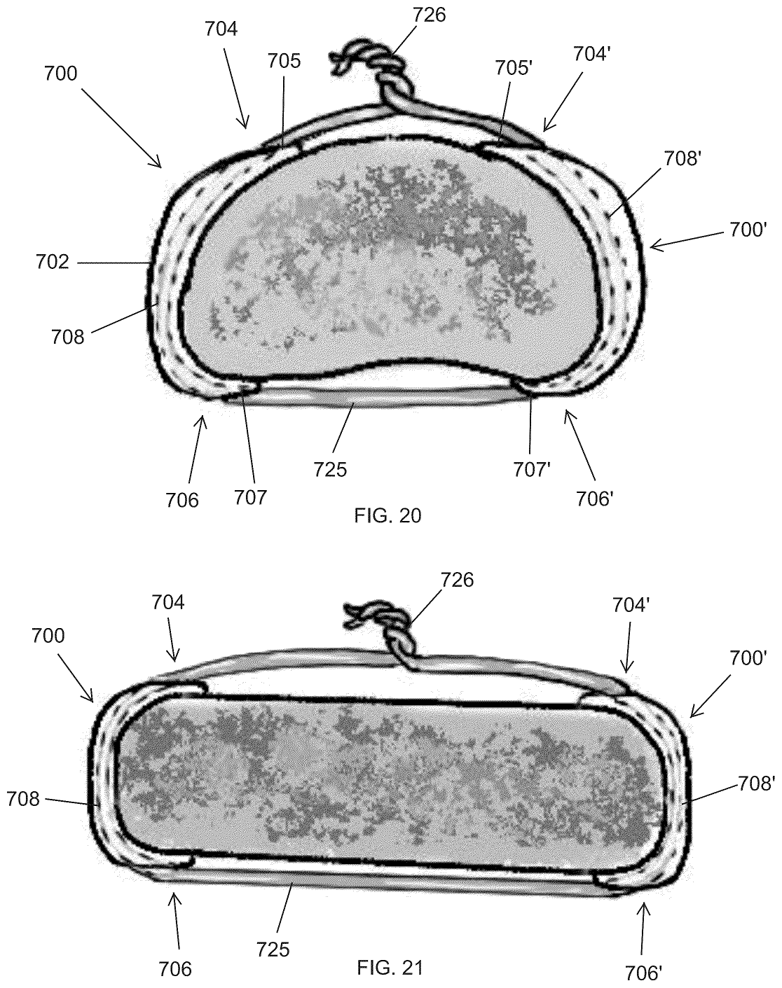

Referring to FIG. 20, in one embodiment, one or more of the pressure distribution implants 700 and the elongated tissue closure device 725 shown and described above in FIGS. 19A and 19B may be used for applying pressure to the sternum during a sternal closure operation.

In one embodiment, a first pressure distribution implant 700 preferably includes a first elongated body 702 having a first end 704 with a first tapered surface 705, a second end 706 with a second tapered surface 707, and a first elongated channel 708 (FIG. 19A) that extends between the first and second ends 704, 706 and along the length of the first elongated body 702. In one embodiment, the first elongated body 702 is made of a flexible/bendable material (e.g., PEEK) so that the first and second ends 704, 706, which extend beyond the sternum at each end of the first elongated body 702, may be bent/flexed over the sternum for conforming to the outer surface of the sternum.

In one embodiment, a second pressure distribution implant 700′ preferably includes a second elongated body 702′ having a first end 704′ with a first tapered surface 705′, a second end 706′ with a second tapered surface 707′, and a second elongated channel 708′ that extends between the first and second ends 704′, 706′ and along the length of the second elongated body 702′. In one embodiment, the second elongated body 702′ is made of a flexible/bendable material (e.g., PEEK) so that the first and second ends 704′, 706′, which extend beyond the sternum at each end of the elongated body 702′, may be bent/flexed over the sternum for conforming to the outer surface of the sternum.

In one embodiment, an elongated tissue closure device 725 (e.g., a wire) is preferably inserted into the first and second elongated channels 708, 708′ of the respective first and second elongated bodies 702, 702′. The free ends of the elongated tissue closure device 725 may be joined together in a twist 726 and tightened for applying pressure to the sternum via the first and second pressure distribution implants 700, 700′ to effectuate sternal closure.

Referring to FIG. 21, in one embodiment, the first and second pressure distribution implants 700, 700′ shown and described above in FIGS. 19A-19B and 20, may be used to close sternums having different cross-sectional shapes, dimensions and/or sizes. For example, the sternum shown in FIG. 21 has a greater width and/or dimension than the sternum shown in FIG. 20.

In FIG. 21, the first and second ends 704, 706 of the first elongated body 702 of the first pressure distribution implant 700, which extend beyond the sternum at each end of the first elongated body 702, are bent/flexed over the sternum. Similarly, the first and second ends 704′, 706′ of the second elongated body 702′ of the second pressure distribution implant 700′, which extend beyond the sternum at each end of the second elongated body 702′, are bent/flexed over the sternum

In one embodiment, an elongated tissue closure device 725 (e.g., a wire) is preferably inserted into the first and second elongated channels 708, 708′ of the respective first and second elongated bodies 702, 702′. The free ends of the elongated tissue closure device 725 may be joined together in a twist 726 and the elongated tissue closing device may be tightened for applying pressure to the sternum via the first and second pressure distribution implants 700, 700′ to effectuate sternal closure.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

What is claimed is:

1. A pressure distribution implant for sternal closure comprising:
an elongated body having a first end, a second end, and a longitudinal axis that extends from the first end to the second end;
the elongated body having top and bottom surfaces that extend along the longitudinal axis;
a plurality of anchors projecting from the top surface of the elongated body and being spaced from one another between the first and second ends of the elongated body, each the anchor including a segment of an elongated channel extending therethrough that is aligned with the longitudinal axis of the elongated body, wherein each the anchor has an opening that is in communication with the segment of the elongated channel associated therewith.

2. The pressure distribution implant as claimed in claim 1, wherein the plurality of anchors define gaps therebetween that overlie the top surface of the elongated body.

3. The pressure distribution implant as claimed in claim 2, wherein the plurality of anchors are evenly spaced from one another between the first and second ends of the elongated body, and wherein each the anchor has a constant length that extends along the longitudinal axis of the elongated body.

4. The pressure distribution implant as claimed in claim 2, wherein the elongated body comprises a flexible body that is more flexible at the location of the gaps and less flexible at the location of the spaced anchors.

5. The pressure distribution implant as claimed in claim 1, wherein the segments of the elongated channel define an elongated channel diameter, and wherein each the opening associated with one of the segments of the elongated channel has a width that is less than the elongated channel diameter.

6. The pressure distribution implant as claimed in claim 5, wherein the segments of the elongated channel that pass through the spaced anchors define tube-shaped hollows that are aligned with the longitudinal axis of the elongated body.

7. The pressure distribution implant as claimed in claim 6, wherein the openings are located at the upper ends of the respective spaced anchors, and wherein the openings are aligned with the longitudinal axis of the elongated body.

8. The pressure distribution implant as claimed in claim 7, further comprising an elongated tissue closure device disposed within the segments of the elongated channel of the spaced anchors, wherein the elongated tissue closure device extends through the spaced anchors and along the longitudinal axis of the elongated body.

9. The pressure distribution implant as claimed in claim 8, wherein the elongated tissue closure device has a cross-sectional diameter that is greater than the width of the openings of the spaced anchors.

10. The pressure distribution implant as claimed in claim 9, wherein the elongated tissue closure device is selected from devices consisting of wires, sutures and polymer bands, and wherein the elongated tissue closure device has a cross-sectional diameter that is less than an inner diameter of the elongated channel.

11. The pressure distribution implant as claimed in claim 1, wherein the spaced anchors are made of a first material that is relatively softer and more flexible than a second material that covers the bottom surface of the flexible body.

12. A pressure distribution implant for sternal closure comprising:
a flexible body having top and bottom surfaces that extend along a longitudinal axis of the flexible body;
a plurality of anchors projecting from the top surface of the flexible body, the anchors being spaced from one another along the longitudinal axis of the flexible body;
the anchors defining a series of gaps therebetween that are located between the anchors, the gaps overlying the top surface of the flexible body and being in alignment with the longitudinal axis of the flexible body;
each the anchor having a segment of an elongated channel extending therethrough, the elongated channel being in alignment with the longitudinal axis of the flexible body;
each the anchor having an outer wall with an opening formed therein that is in communication with the respective segment of the elongated channel associated therewith.

13. The pressure distribution implant as claimed in claim 12, wherein the openings of the respective anchors are aligned with the longitudinal axis of the flexible body.

14. The pressure distribution implant as claimed in claim 13, wherein the segments of the elongated channel have cross-sectional diameters, and wherein the openings have widths that are smaller than the cross-sectional diameters of the respective segments of the elongated channel.

15. The pressure distribution implant as claimed in claim 14, further comprising an elongated tissue closure device being secured over the top surface of the flexible body by the spaced anchors, the elongated tissue closure device extending through the segments of the elongated channel of the respective anchors for being aligned with the longitudinal axis of the flexible body.

16. The pressure distribution implant as claimed in claim 12, wherein the flexible body has first and second flexible ends that are configured for being bent for conforming to the shape of an outer surface of a sternum.

17. The pressure distribution implant as claimed in claim 12, further comprising:
a second flexible body having top and bottom surfaces that extend along a second longitudinal axis that intersects with the first longitudinal axis of the first flexible body;
a plurality of second anchors projecting from the top surface of the second flexible body, the second anchors being spaced from one another along the second longitudinal axis of the second flexible body;
the second anchors defining a series of second gaps located between the respective second anchors and that are in alignment with the second longitudinal axis of the second flexible body;
each the second anchor having a segment of a second elongated channel extending therethrough that is in alignment with the second longitudinal axis of the second flexible body;
each the second anchor having an outer wall with a second opening formed therein that is in communication with one of the segments of the second elongated channel extending therethrough.

18. The pressure distribution implant as claimed in claim 17, further comprising an elongated tissue closure device being secured over the top surface of the second flexible body by the second spaced anchors, the elongated tissue closure device extending through the segments of the second elongated channel and being aligned with the second longitudinal axis of the second flexible body.

19. The pressure distribution implant as claimed in claim 17, wherein the first and second flexible bodies cross one another and define an implant structure having an X-shaped configuration.

20. The pressure distribution implant as claimed in claim 19, further comprising a pin that pivotally interconnects the first and second flexible bodies for selectively changing the angle between the first longitudinal axis of the first flexible body and the second longitudinal axis of the second flexible body.

21. A pressure distribution implant for sternal closure comprising:
a flexible body having a first end, a second end, and a longitudinal axis that extends from the first end to the second end;
the flexible body having top and bottom surfaces that extend along the longitudinal axis;
a plurality of anchors projecting from the top surface of the flexible body and being spaced from one another between the first and second ends of the flexible body, each the anchor including a segment of an elongated channel that that is aligned with the longitudinal axis of the flexible body, each the anchor having an opening that is in communication with the segment of the elongated channel associated therewith;
an elongated tissue closure device secured over the top surface of the flexible body by the anchors, the elongated tissue closure device extending through the segments of the elongated channel that is aligned with the longitudinal axis of the flexible body, wherein the elongated tissue closure device has a cross-sectional diameter that is greater than a width of the openings of the respective anchors.

22. The pressure distribution implant as claimed in claim 21, wherein the plurality of spaced anchors define gaps therebetween that overlie the top surface of the flexible body and that are located between the spaced anchors, wherein the flexible body is relatively more flexible at the location of the gaps and relatively less flexible at the location of the spaced anchors.

23. The pressure distribution implant as claimed in claim 22, wherein the flexible body comprises a biocompatible polymer material, and wherein the spaced anchors are made of a first material that is softer and more flexible than a second material that covers the bottom surface of the flexible body.

24. The pressure distribution implant as claimed in claim 21, wherein the elongate tissue closure device comprises devices selected from the group consisting of wires, sutures, and polymer bands.
